(19) United States
(12) Patent Application Publication
(10) Pub. No.: US 2026/0315287 A1
(43) Pub. Date: Oct. 15, 2026

(54) ALGINATE TAMPON AND PRODUCTION METHOD

(71) Applicant: VYLD GMBH, Berlin (DE)

(72) Inventor: Ines SCHILLER, Berlin (DE)

(73) Assignee: VYLD GMBH, Berlin (DE)

(21) Appl. No.: 19/104,257

(22) PCT Filed: Aug. 18, 2023
(86) PCT No.: PCT/EP2023/072867

§ 371 Date: Feb. 17, 2025

(30) Foreign Application Priority Data
Aug. 19, 2022 (EP) 22191331.2
Aug. 19, 2022 (EP) 22191334.6

(51) Int. Cl. A61F 13/20; A61F 13/84; A61K 9/00; A61K 31/734; A61L 15/24; A61L 15/28

(52) U.S. Cl. CPC A61F 13/2051; A61F 13/2088; A61F 13/2094; A61F 13/8405; A61K 9/0036; A61K 31/734; A61L 15/24; A61L 15/28

(57) ABSTRACT

The present invention relates to a tampon at least partially made of alginate and a production method thereof.

### FIELD

The present invention relates to a tampon at least partially made of alginate and a production method thereof.

### BACKGROUND

The objective of the present invention is to provide means and methods for a bio-degradable tampon with improved properties. This objective is attained by the subject-matter of the independent claims of the present specification, with further advantageous embodiments described in the dependent claims, examples, figures and general description of this specification.

### SUMMARY OF THE INVENTION

The majority of tampons sold are made of rayon, or a blend of rayon and cotton, along with synthetic fibres. The present invention relates to a tampon which is (at least partially) made of alginate from seaweed.

Once inserted correctly, a tampon is held in place by the vagina and expands as it soaks up menstrual fluid. However, in addition to menstrual blood, a conventional tampon also absorbs the vagina's natural lubrication and bacteria, which can change the normal pH, leading to irritations and increasing the risk of infections, including serious infections from the bacterium Staphylococcus aureus, which can lead to toxic shock syndrome (TSS).

The tampon of the present invention is not only bio-degradable on land and in water, but also minimizes the risk for TSS and other infections.

### TERMS AND DEFINITIONS

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising”, “having”, “containing”, and “including”, and other similar forms, and grammatical equivalents thereof, as used herein, are intended to be equivalent in meaning and to be open-ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a”, “or” and “the” include plural referents unless the context clearly dictates otherwise.

“And/or” where used herein is to be taken as specific recitation of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art (e.g., in cell culture, molecular genetics, nucleic acid chemistry, hybridization techniques and biochemistry, organic synthesis). Standard techniques are used for molecular, genetic, and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term tampon in the context of the present specification relates to a product which is placed internally, inside of the vaginal canal. A tampon may be used to prevent or treat a medical indication, or during menstruation to absorb blood and vaginal secretions.

The term alginate in the context of the present specification relates to a polysaccharide of the chemical structure [D-ManA(β1→4)L-GulA(α1→4)]ₙ. This means, alginate is composed of β-(1-4) linked d-mannuronic acid (=“M”) units and β-(1-4)-linked l-guluronic acid units (=“G”) units. Alginate is a naturally occurring, edible polysaccharide found in brown algae and is the source of the seaweed's high flexibility strength and water absorbency. Alginate is present as a salt. In most cases, alginate comprises the counterion Ca²⁺ and/or Na⁺. In the context of the present invention, the term “alginate” comprises the polysaccharide alginate and also counterions, and water to a certain extent.

The term tampon absorption body or core in the context of the present specification relates to the part of the tampon with high liquid absorption allowing the tampon to absorb and hold menstrual fluids provided by hydrophilic fibres or other highly liquid absorptive materials.

The term tampon overwrap or liner in the context of the present specification relates to the outer layer of the tampon (partially) surrounding the tampon core to facilitate insertion and removal of tampon and preventing fibre loss (linting).

The term tampon ribbon or tampon blank in the context of the present specification relates to the carded fibres for the tampon core before further processing.

The term tampon pledget in the context of the present specification relates to the tampon precursor made from the tampon ribbon before pressing the tampon in shape.

The term M/G ratio (unit/unit) in the context of the present specification relates to the ratio between d-mannuronic acid (M) and l-guluronic acid (G) inside the polymer alginate. The ratio is determined as unit per unit meaning that the number of M and G units are counted and the number of M units is divided by the number of G units.

The term weight ratio in the context of the present specification relates to a ratio between different ions (calcium and sodium). The weight of calcium ions and the weight of sodium ions is determined and then these weights are divided to yield the weight ratio of calcium to sodium (or vice versa).

The term highly viscous in the context of the present specification relates to a viscosity of ≥3 mPa·s.

Any patent document cited herein shall be deemed incorporated by reference herein in its entirety.

### DETAILED DESCRIPTION OF THE INVENTION

A first aspect of the invention relates to a tampon (intravaginal device). The tampon absorption body (core) comprises 25%-100% (w/w) of alginate fibre. The tampon does not comprise polyolefin fibre, particularly a liner made of polyolefin fibre (plastic fibre, for example polypropylene, polyethylene).

In certain embodiments, the tampon absorption body comprises ≥30% (w/w) alginate. In certain embodiments, the tampon absorption body comprises ≥35% (w/w) alginate. In certain embodiments, the tampon absorption body comprises ≥40% (w/w) alginate. In certain embodiments, the tampon absorption body comprises ≥45% (w/w) alginate. In certain embodiments, the tampon absorption body comprises ≥50% (w/w) alginate. In certain embodiments, the tampon absorption body comprises ≥55% (w/w) alginate. In certain embodiments, the tampon absorption body comprises ≥60% (w/w). In certain embodiments, the tampon absorption body comprises ≥65% (w/w). In certain embodiments, the tampon absorption body comprises ≥70% (w/w). In certain embodiments, the tampon absorption body comprises ≥75% (w/w). In certain embodiments, the tampon absorption body comprises ≥80% (w/w). In certain embodiments, the tampon absorption body comprises ≥85% (w/w). In certain embodiments, the tampon absorption body comprises ≥90% (w/w). In certain embodiments, the tampon absorption body comprises ≥95% (w/w) alginate. In certain embodiments, the tampon absorption body consists of 100% (w/w) alginate.

In certain embodiments, the tampon absorption body, in addition to alginate, comprises ≤75% (w/w) of an additional absorptive material selected from the group comprising cotton, normal viscose and high absorbency viscose (for example trilobal viscose, hollow fibers viscose) (rayon), Lyocell, Bamboo fibre, Eukalyptus fibre and/or hemp fibre. In certain embodiments, the additional absorptive material is selected from the group comprising cotton and/or viscose. In certain embodiments, the additional absorptive material is cotton. In certain embodiments, the viscose has a normal/high absorbency ratio of 80/20 (weight/weight).

In certain embodiments, the alginate is composed of β-(1-4) linked d-mannuronic acid (=“M”) units and β-(1-4)-linked l-guluronic acid units (=“G”) units, and the alginate is characterized by an M/G ratio (unit/unit) of 0.4-1.5. In certain embodiments, the alginate is characterized by an M/G ratio (unit/unit) of 0.45-0.9. In certain embodiments, the alginate is characterized by an M/G ratio (unit/unit) of ~0.5.

In certain embodiments, the alginate fibre is a sodium alginate fibre, a calcium alginate fibre, or a mixed alginate fibre. In certain embodiments, the alginate fibre is a mixed fibre.

In certain embodiments, the alginate is present as a mixed salt comprising sodium and calcium ions, and the alginate is characterized by a weight ratio of calcium to sodium between 1-200. In certain embodiments, the alginate is characterized by a weight ratio of calcium to sodium between 2.5-120. In certain embodiments, the alginate is characterized by a weight ratio of calcium to sodium between 10-50. In certain embodiments, the alginate is characterized by a weight ratio of calcium to sodium between 17-35. In certain embodiments, the alginate is characterized by a weight ratio of calcium to sodium ~25.

In certain embodiments, the alginate is characterized by an M/G ratio (unit/unit) of ~0.5 and a calcium to sodium weight ratio of 17-35. The medium G-content (M/G ratio ~0.5) leads to a relatively high fibre strength as well as good liquid absorbency.

In certain embodiments, the tampon comprises a bio-degradable liner (overwrap). In certain embodiments, the bio-degradable liner is composed of polybutylene succinate (PBS), natural gum and/or synthetically modified alginate. In certain embodiments, the tampon comprises a water-sprayed alginate liner.

In certain embodiments, the alginate is obtained from algae of the class Phaeophyceae (brown seaweed). In certain embodiments, the alginate is obtained from algae of a genus selected from the group comprising Macrocystis, Laminaria, Lessonia, Ascophyllum, Saccharina, Sargassum, Fucus.

In certain embodiments, the outer surface of the tampon pledget is characterized by a ratio of non-bundled fibre strands to bundled fibre strands of at most (≤) 2.3, particularly of at most (≤) 1.0. The ratio is measured in comparison to the core of the tampon.

### SEM Method

The ratio of non-bundled fibre strands to bundled fibre strands may be determined via a method comprising the steps:
Acquiring at least five reference scanning electron microscope (SEM) images of the core of said tampon, (particularly recorded at a magnification of 480× and at equal distances over a longitudinal length of 4 cm (wherein longitudinal refers to the orientation of the tampon)),
wherein the reference SEM images are segmented to obtain reference segmented images comprising foreground and background;
Computing a local fibre thickness of non-bundled fibre strands within the reference segmented images to obtain reference local thickness images and a base line (or reference value) of fibre thickness of the non-bundled fibre strands;
Acquiring at least five (outer) surface SEM images of the (outer) surface of the tampon pledget, (particularly recorded at a magnification of 480× and at equal distances over a longitudinal length of 4 cm (wherein longitudinal refers to the orientation of the tampon)),
and wherein said (outer) surface SEM images are segmented to obtain (outer) surface segmented images;
Computing a local fibre thickness of the (outer) surface SEM images within the (outer) surface segmented images to obtain (outer) surface local thickness images and a base line (or reference value) of fibre thickness of the non-bundled fibre strands;
Computing a ratio of non-bundled fibre strands to bundled fibre strands within the (outer) surface of the tampon pledget via the (outer) surface local thickness images, wherein an average of the area under the curve (AUC) of local fibre thicknesses within the (outer) surface local thickness images below the reference value (base line) is divided by an average of the AUC of local fibre thicknesses above the reference value of fibre thickness within the (outer) surface local thickness images.

The method involves utilizing a scanning electron microscope (SEM) to capture an image of untreated fibres of the tampon core. As common, SEM images are grey scale. Subsequently, the image is subjected to segmentation, resulting in a first segmented image. Local fibre thickness measurements are then derived from the first (also called “reference”) segmented SEM image. A median value and standard deviation of the fibre thicknesses are computed, and their summation establishes a baseline for the untreated fibre thickness.

Continuing the method, second (also called “outer surface”) SEM images are acquired from the same sample at the outer surface. Like the previously described process, these second SEM images undergo segmentation to produce one or more second segmented images. Further analysis involves calculating the local fibre thicknesses from these second segmented images.

To ascertain the ratio of non-bundled fibre strands to bundled fibre strands, the local fibre thickness values from the second segmented image below the baseline of untreated fibres are juxtaposed with the fibres or fibre bundles in the same image that lie above the untreated fibre baseline. The relationship between the thicknesses of fibres and fibre bundles as a function of the applied solution to the fibres exhibits an essentially linear decline.

### Image Segmentation

There are many methods for image segmentation known in the art. Without limitation, some methods are named below that are suitable for use in the method described above. If in doubt, the Otsu's method is used for quantification.

Thresholding is a straightforward image segmentation technique that involves separating objects from the background based on pixel intensity. A threshold value is set, and pixels with intensities above the threshold are assigned to one segment, while pixels with intensities below the threshold are assigned to another segment. This method is particularly effective when the foreground and background have distinct intensity ranges. There are different types of thresholding methods, such as global thresholding, adaptive thresholding, and Otsu's thresholding, which automatically determine the threshold value based on the image histogram. Region growing is a method where segments are grown from seed points or regions based on certain predefined criteria. It begins with one or more seed points and iteratively adds neighboring pixels that meet specific similarity criteria. These criteria could include similarity in intensity, color, texture, or other feature attributes. The growing process continues until no more pixels satisfy the criteria. Region growing can be sensitive to seed point selection and criteria definition, but it is useful for segmenting objects with uniform characteristics. Edge-based segmentation involves detecting boundaries or edges of objects within an image. Once edges are detected, techniques like edge linking and contour following are used to connect edge points and form closed contours. These contours define the boundaries of segmented objects. Edge-based segmentation is suitable for images with well-defined object edges.

In particular one of the following methods is preferred for carrying out image segmentation. K-Means clustering groups pixels based on their intensity values into clusters, effectively segmenting the image into distinct regions. Each cluster represents a segment or object in the image.

Watershed segmentation treats the grayscale image as a topographic landscape, where intensity values correspond to heights. Minima in this landscape represent potential segment boundaries. Watershed lines are applied to separate regions, forming distinct segments. This algorithm is particularly useful for segmenting objects with well-defined boundaries.

Thresholding techniques separate pixels into segments based on intensity threshold values. Otsu's Method automatically determines an optimal threshold that separates the image into foreground and background based on maximizing inter-class variance. Adaptive thresholding adjusts the threshold locally to account for varying illumination conditions in different parts of the image. Otsu's method is a binary version of the K-Means clustering method and the particularly preferred method for image segmentation herein.

Graph-Cut segmentation treats grayscale image segmentation as a graph optimization problem. It constructs a graph where nodes represent pixels and edges represent pairwise similarities. By optimizing the cut in the graph, pixels are divided into segments. Graph-Cut can be adapted for grayscale images to achieve accurate segmentation results.

### Local Thickness

The local thickness of fibres is calculated by computing for each pixel the radius of the largest sphere that both engulfs the pixel and fits entirely within the foreground.

Other methods are also known in the art (but only mentioned herein for reference) such as morphological thinness measurement, a technique that leverages morphological operations to estimate the local thickness of linear structures in a grayscale image. The algorithm of the selected method works as follows:

Skeletonization: First, the image is skeletonized, which involves reducing the width of the linear structures to a single-pixel thickness while preserving their connectivity.

Distance Transform: A distance transform is applied to the skeletonized image. This transform assigns each pixel a value corresponding to its distance from the nearest background pixel. This step effectively measures the thickness of the structures.

Local Maxima Extraction: Local maxima of the distance transform are identified. These maxima correspond to points on the skeletonized image where the thickness is greatest.

Local Thickness Calculation: The local thickness at each local maximum is calculated by considering the distance values of neighboring pixels. This provides an estimate of the thickness of the fibres at that point.

Another known method for computing the local fibre thickness is the Hough Transform-based Method (only for reference):

The Hough transform is a technique often used for detecting lines or linear structures in an image. It can also be adapted to estimate the local thickness of fibres. The algorithm proceeds as follows:

Edge Detection: Edges in the grayscale image are detected using edge detection algorithms, such as the Sobel operator or Canny edge detector. This highlights the linear structures.

Hough Transform: The Hough transform is applied to the edge-detected image to detect lines (fibres) and estimate their parameters, such as angle and position.

Local Thickness Estimation: For each detected line (fibre), the local thickness is estimated by measuring the distance between nearby parallel lines. This distance corresponds to the width of the fibre at that point.

Post-Processing: The local thickness estimates can be further refined through post-processing steps, such as filtering out noise or smoothing the thickness values.

A second aspect of the invention relates to alginate for use in prevention of toxic shock syndrome (TSS), wherein the alginate is administered in the form of a tampon as specified in the first aspect.

A third aspect of the invention relates to alginate for use in prevention of vaginal infection, wherein the alginate is administered in the form of a tampon as specified in the first aspect. In certain embodiments, the vaginal infection is vaginal yeast infection or infection with E. coli.

A further aspect of the invention relates to alginate for use in enhancement of Lactobacillus gasseri and Lactobacillus crispatus growth in the vaginal microbiome, wherein the alginate is administered in the form of a tampon as specified in the first aspect.

A further aspect of the invention relates to a use of a tampon according to the first aspect for absorption of mucosal cells and highly viscous fluid.

A further aspect of the invention relates to a method for production of a tampon, said method comprising the steps:
a. providing a longitudinally extending strip of fibre material as a tampon ribbon (tampon blank), wherein the fibre material is a fibre blend comprising 25%-100% (w/w) alginate, and 0-75% (w/w) other bio-degradable fibre (selected from the group comprising cotton, viscose and high absorbency viscose (for example trilobal viscose, hollow fibers viscose) (rayon), Lyocell, Bamboo fibre, Eukalyptus fibre and/or hemp fibre);
b. a spraying step;
c. in a rolling step, rolling the tampon ribbon yielding a non-shaped tampon pledget;
d. in a pressing step, pressing the non-shaped tampon pledget via a press to form a shaped tampon pledget;
e. in a head-forming step, forming a head for the shaped tampon pledget using a heated round head and a pointed head shaper to yield a tampon;
   characterized in that in the spraying step, one part of the tampon ribbon is sprayed with water (finely dispersed water), and the water-sprayed part forms the outer surface of the tampon pledget.

A further aspect of the invention relates to a method for production of a tampon, said method comprising the steps:
a. providing a longitudinally extending strip of fibre material as a tampon ribbon (tampon blank);
b. a spraying step;
c. in a rolling step, rolling the tampon ribbon yielding a non-shaped tampon pledget, wherein an outer surface of the tampon pledget or the tampon overwrap comprises 10%-100% (w/w) alginate (wherein the outer surface amounts to ~2-10% (w/w) of the tampon pledget);
d. in a pressing step, pressing the non-shaped tampon pledget via a press to form a shaped tampon pledget;
e. in a head-forming step, forming a head for the shaped tampon pledget using a heated round head and a pointed head shaper to yield a tampon;
   characterized in that in the spraying step, one part of the tampon ribbon is sprayed with water (finely dispersed water), and the water-sprayed part forms the outer surface of the tampon pledget.

In certain embodiments, the sprayed water is deionized water.

In certain embodiments, the sprayed water comprises ions.

In certain embodiments, 0.2-0.4 ml of water is used per tampon. In certain embodiments, ~0.32 ml of water is used per tampon. The amount of sprayed water refers to a tampon with 254 mm length×50 mm width of the tampon ribbon with the ribbon having ~10 g/m weight. The amount of water will be adjusted for smaller or larger tampons.

In certain embodiments, the percentage of water being used per tampon is 5-20% (v/w), particularly wherein the percentage is ~10% (v/w). In certain embodiments, the water-sprayed part of the tampon ribbon is ~⅓ of the length of the tampon ribbon. In certain embodiments, a sodium ion concentration of said water is 0.43-2.2 mol/L.

In certain embodiments, the press is a radial press.

In certain embodiments, the press is a lateral press.

In certain embodiments, the temperature of the press in the pressing step is ≤200° C. In certain embodiments, the temperature of the press in the pressing step is ≤120° C.

In certain embodiments, the spraying step is performed ≤one minute before the rolling step.

In certain embodiments, the water-sprayed part of the tampon ribbon is 5 cm to 9 cm in length.

Alginate fibres must be stable in vaginal environments of low pH 3.8-4.5 (acidic). A study showed that calcium alginate fibres remain stable and durable at pH levels as low as 3.4 (Lim et al., Journal of Environmental Chemical Engineering, Volume 9, Issue 5, October 2021).

The tampons of the invention are produced using conventional tampon production machines which allows to achieve industrial level processing properties similar to those of viscose. Alginate fibres are hygroscopic with a high moisture regain similar or even better to conventional tampon fibres. Good fibre processing properties also include minimal fibre loss during carding and opening, high fibre crimp, optimal fibre diameter and low brittleness.

The biodegradability of alginate fibres has not yet been extensively researched. However, there is research on the biodegradability of alginate films and seaweed plastics. This information is a good measure of what should be expected for alginate fibres. For example, bioplastics made from polysaccharides extracted from red seaweed are biodegrade in the soil in only 4-6 weeks (Lim et al., Journal of Environmental Chemical Engineering, Volume 9, Issue 5, October 2021). This process is similar to viscose (4-6 weeks) and much faster than cotton (minimum 5 months).

Performance: Alginate fibres have excellent gelling properties preventing dehydration and contributing to the overall performance of the tampon of the invention. Alginate fibres also have better absorption than cotton and viscose fibres. Alginate fibres are capable of absorbing 12-25× their own weight in physiological saline solution according to NWSP 010.1.R0 (15)b. Although the absorption capacity in Syngina testing according to NWSP350.1.R1 (15) is lower (270-445%) than that of viscose (400-570%) and cotton (630%), it is more suitable for application in a tampon. The absorption capacity needs to be high enough to absorb and retain menstrual fluid, but low enough as to prevent dehydration of the vaginal mucous membrane. The combination of the high absorption capacity, the gelling effect and the softness of the alginate fibres make them ideal for tampon production.

A further aspect of the invention relates to a tampon obtained by the method according to the previous aspect.

In certain embodiments, the tampon is characterized by having no overwrap and wherein the tampon is capable of losing only a medium to low (moderate to minimal) amount of fibre in a saline shedding test.

In certain embodiments, the tampon is characterized by having no overwrap and wherein the tampon is capable of losing only a medium to low (moderate to minimal) amount of fibre in a dry shedding test.

In certain embodiments, the outer surface of the tampon pledget is characterized by a ratio of non-bundled fibre strands to bundled fibre strands of at most 2.3, particularly of at most 1.0.

### Saline Shedding Method:

Immerse tampon in saline for 5 minutes;
Remove tampon for 3 seconds and immerse for a further 3 seconds (repeated 3 times);
Immerse tampon in saline for a further 5 minutes;
Remove tampon and visually assess integrity of wet tampon;
Filter saline to quantify the shed fibre.

### Dry Shedding Method:

Manually wrapping black test sticker around tampon without applying pressure;
Applying pressure on the wrapped tampon using Syngina tester;
Removing the test sticker from the tampon without applying pressure;
Visually assessing the amount of fibre adhering to the sticker.

A reference picture of shedding is depicted in FIGS. 8 and 9.

### Medical Treatment

Similarly, within the scope of the present invention is a method of preventing vaginal infection or TSS in a patient in need thereof, comprising administering to the patient an alginate tampon according to the above description.

The invention further encompasses, as an additional aspect, the use of alginate as identified herein for use in a method of manufacture of a medicament for the prevention of vaginal infection or TSS.

The invention further encompasses the following items.

### Items

1. A tampon, wherein
   a tampon absorption body comprises 25%-100% (w/w) of alginate fibre;
   the tampon does not comprise polyolefin fibre.
2. The tampon according to item 1, wherein the tampon absorption body comprises ≥50% (w/w) alginate, particularly the tampon absorption body comprises ≥65% (w/w), ≥70% (w/w), ≥75% (w/w), ≥80% (w/w), ≥85% (w/w), ≥90% (w/w), ≥95% (w/w) alginate, more particularly the tampon absorption body consists of 100% (w/w) alginate.
3. The tampon according to any one of the preceding items, wherein the tampon absorption body, in addition to alginate, comprises ≤75% (w/w) of an additional absorptive material selected from the group comprising cotton, viscose and high absorbency viscose (for example trilobal viscose, hollow fibers viscose) (rayon), Lyocell, Bamboo fibre, Eukalyptus fibre and/or hemp fibre particularly an additional absorptive material selected from the group comprising cotton and/or viscose, more particularly wherein the additional absorptive material is cotton.
4. The tampon according to any one of the preceding items, wherein the alginate is composed of β-(1-4) linked d-mannuronic acid (=“M”) units and β-(1-4)-linked I-guluronic acid units (=“G”) units, and the alginate is characterized by an M/G ratio (unit/unit) of 0.4-1.5, particularly the alginate is characterized by an M/G ratio (unit/unit) of 0.45-0.9, more particularly the alginate is characterized by an M/G ratio (unit/unit) of ~0.5.
5. The tampon according to any one of the preceding items, wherein the alginate is present as a mixed salt comprising sodium and calcium ions, and the alginate is characterized by a weight ratio of calcium to sodium between 1-200, particularly the alginate is characterized by a weight ratio of calcium to sodium between 2.5-120, more particularly the alginate is characterized by a weight ratio of calcium to sodium between 10-50, even more particularly between 17-35, most particularly the alginate is characterized by a weight ratio of calcium to sodium ~25.
6. The tampon according to any one of the preceding items, wherein the alginate is characterized by an M/G ratio (unit/unit) of ~0.5 and a calcium to sodium weight ratio of 17-35.
7. The tampon according to any one of the preceding items, wherein the tampon comprises a bio-degradable liner.
8. The tampon according to item 7, wherein the bio-degradable liner is composed of polybutylene succinate.
9. The tampon according to any one of the preceding items, wherein the alginate is obtained from algae of the class Phaeophyceae (brown seaweed), particularly from algae of a genus selected from the group comprising Macrocystis, Laminaria, Lessonia, Ascophyllum, Saccharina, Sargassum, Fucus.
10. Alginate for use in prevention of toxic shock syndrome (TSS), wherein the alginate is administered in the form of a tampon as specified in any one of items 1 to 9.
11. Alginate for use in prevention of vaginal infection, wherein the alginate is administered in the form of a tampon as specified in any one of items 1 to 9, particularly wherein the vaginal infection is vaginal yeast infection or infection with E. coli.
12. Alginate for use in enhancement of Lactobacillus gasseri and Lactobacillus crispatus growth in the vaginal microbiome, wherein the alginate is administered in the form of a tampon as specified in any one of items 1 to 9.
13. Use of a tampon according to any one of the preceding items 1 to 9 for absorption of mucosal cells and highly viscous fluid.
14. A method for production of a tampon, said method comprising the steps:
   a. providing a longitudinally extending strip of fibre material as a tampon ribbon, wherein the fibre material is a fibre blend comprising 25%-100% (w/w) alginate, and 0-75% (w/w) other bio-degradable fibre;
   b. a spraying step;
   c. in a rolling step, rolling the tampon ribbon yielding a non-shaped tampon pledget;
   d. in a pressing step, pressing the non-shaped tampon pledget via a press to form a shaped tampon pledget;
   e. in a head-forming step, forming a head for the shaped tampon pledget using a heated round head and a pointed head shaper to yield a tampon;
characterized in that in the spraying step, one part of the tampon ribbon is sprayed with water, and the water-sprayed part forms the outer surface of the tampon pledget.
15. The method according to item 14, wherein 0.2-0.4 ml of water is used per tampon, particularly ~0.32 ml of water is used per tampon.
16. The method according to any one of the preceding items 14 to 15, wherein the press is a radial press.
17. The method according to any one of the preceding items 14 to 16, wherein the press is a lateral press.
18. The method according to any one of the preceding items 14 to 17, wherein the temperature of the press in the pressing step is ≤200° C., particularly the temperature of the press in the pressing step is ≤120° C.
19. The method according to any one of the preceding items 14 to 18, wherein the spraying step is performed ≤one minute before the rolling step.
20. The method according to any one of the preceding items 14 to 19, wherein the water-sprayed part of the tampon ribbon is 5 cm to 9 cm in length.

The invention is further illustrated by the following examples and figures, from which further embodiments and advantages can be drawn. These examples are meant to illustrate the invention but not to limit its scope.

### DESCRIPTION OF THE FIGURES

FIG. 1 Growth of S. aureus.

FIG. 2 Mean growth of S. aureus.

FIG. 3 TSST-1 toxin production.

FIG. 4 Mean TSST-1 toxin production.

FIG. 5 Evaluation of CFU/ml at 0 h, 24 h and 96 h for L. crispatus.

FIG. 6 Evaluation of CFU/ml at 0 h, 24 h and 96 h for L. gasseri.

FIG. 7 Scanning electron microscope images of tampons with different Ca/Na ratios. Magnification: 480.

FIG. 8 Saline shedding test results.

FIG. 9 Dry shedding test results.

FIG. 10 Upper panel: segmented images according to the SEM method. Lower panel: Local thickness of non-bundled fibres (left) and of partially bundled fibres (middle and right).

FIG. 11 Diameter density of non-bundled fibres (0%) and of partially bundled fibres (30% and 50%).

FIG. 12 AUC of 30% bundled fibres.

### EXAMPLES

### Example 1: Absorbency

Comparison of tampon Syngina testing according to NWSP350.1.R1 (15) and fibre liquid absorptive capacity (LAC) immersion testing according to NWSP 010.1.R0 (15)b of alginate fibres with cotton and viscose.

TABLE 1

| | | liquid absorptive | liquid absorptive |
|---|---|---|---|
| | Ca conc | vapacity - | capacity - Immersion |
| Fibre | (g/kg) | Syngina test [%] | [g/g] |
| Fibre 1 | 59.97 | 267.6 | 18 |
| Fibre 2 | 68.34 | 100.2 | 10.6 |
| Fibre 3 | 89.81 | 125.4 | 13.2 |
| Fibre 4 | 65.82 | 117.4 | 13.3 |
| Fibre 5 | 61.95 | 179 | 15.8 |
| Fibre 6 | 61.64 | 118.8 | 9.1 |
| Fibre 7 | 46.99 | 140 | 19 |
| Fibre 8 | 39.95 | 250.9 | 19 |
| Fibre 9 | 64.28 | 108.8 | 12.8 |
| Fibre 10 | n.a. | 82.28 | 25.7 |
| Fibre 11 | 68.27 | 308.6 | 12 |
| Cotton | n.a. | 634.9 | 25.6 |
| Viscose (High | n.a. | 573.1 | 21.5 |
| Absorbency) | | | |
| Viscose normal | n.a. | 401 | — |

### Example 2: M/G & Calcium/Sodium Ratio

The properties of alginate fibres are greatly affected by the final calcium/sodium ratio which is determined by the M/G ratio, in particular the G-units.

The large calcium ions can only bind to G-units due to hindrance reasons whereas sodium ions can bind to both G- and M-units. In the coagulation bath calcium ions exchange with sodium ions calcium ions replace the sodium ions on the G-units. The final amount of calcium ions bound on the alginate molecule determines how stiff the molecule becomes. Calcium ions create cross-linking in the polymer resulting in an egg-box model: one calcium ion (Ca²⁺) can bind to two alginate monomers by cross-linking. This reduces the flexibility of the molecule. The more G-units there are in an alginate molecule, the more calcium ions can bind to it and the more stiff (less flexible) the gel becomes which leads to stiffer (stronger) alginate fibres.

To obtain the desired properties of alginate fibres for tampon production the fibres must have the perfect balance between stiff (strong) and flexible (soft). For this a medium M/G ratio must be considered.

TABLE 2

| | | Performance | Processability |
|---|---|---|---|
| | | (absorbency | (fiber loss during |
| Fibre | Ca/Na ratio | between 9-12 g) | carding [%]) |
| Fibre 1 | 109.51 | Medium | 23.1 |
| Fibre 2 | 63.30 | Good | 20.1 |
| Fibre 3 | 35.53 | Very good | 2.9 |
| Fibre 4 | 29.37 | Very good | 17.1 |
| Fibre 5 | 20.73 | Good | n.a. |
| Fibre 6 | 17.63 | Very good | 29.4 |
| Fibre 7 | 2.5 | Good | n.a. |

TABLE 6

| | M/G | LAC % | Gel blocking |
|---|---|---|---|
| Fiber 1 | 0.62 | 267.6 | no |
| Fiber 3 | 1.59 | 179.0 | yes |
| Fiber 3 | 3.00 | 108.8 | yes |

M/G < 1.5: prevented or delayed gel blocking leading to higher liquid absorption capacity.
M/G > 1.5: more fiber swelling causes gel blocking sooner impeding liquid absorption.

### Example 3: TSS Results

### TSS

Here the inventors tested the alginate fibres Fibre 1, Fibre 11, Fibre 8 for TSST-1 and compared it to a control material (Playtex Regular).

Stationary Flask Method for TSS S. aureus Apr. 20, 2022
2 grams of each fibre were weighted and placed in 125 ml Erlenmeyer Flasks. 3 flasks were used per sample type.
10 ml of Todd Hewitt broth was added to each sample plus 10 ml to a no product control flask.
All were done in triplicate with a starting inoculum of 7.8E+06/ml. This means 7.8 times 10 to the 6th/ml.
All flasks were incubated in 5% CO2 for 24 hours. Then, liquid samples were collected by squeezing through syringes or simply pipetting in the case of the no product control.
Plate counts on blood agar were done to assess S. aureus MN8 growth.
Western immunoblot was used to assess TSST-1.

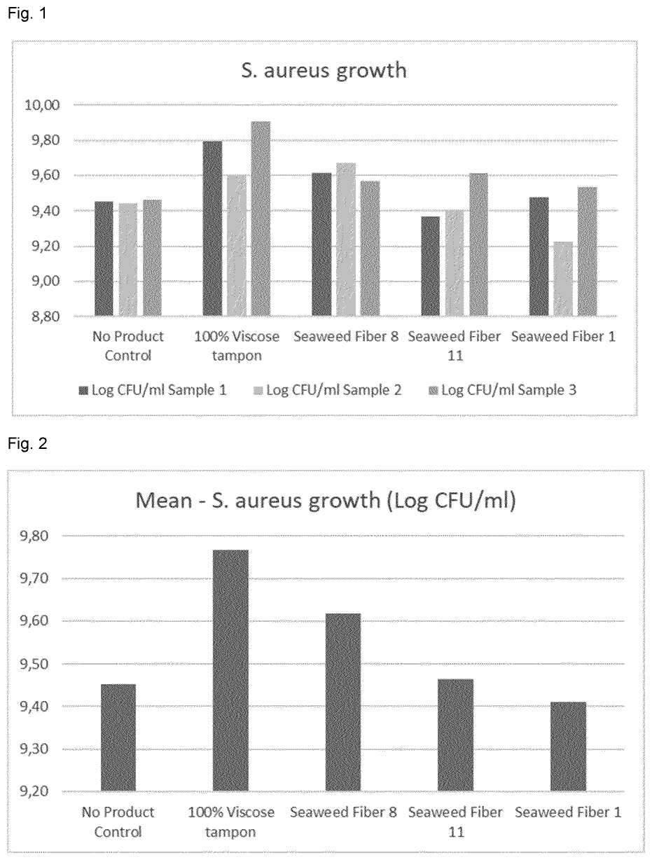

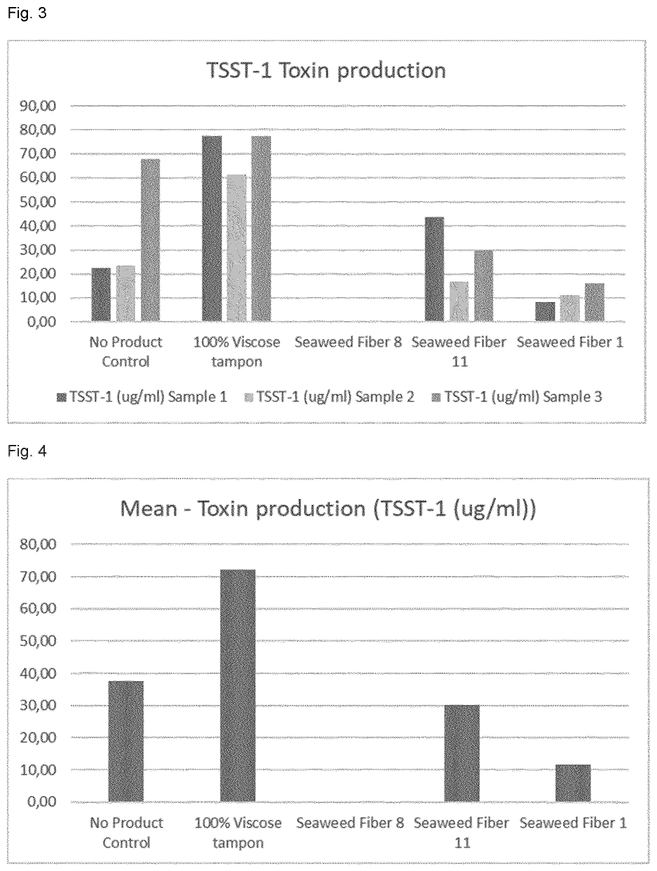

The data is shown below in FIG. 1-4.

There was very little difference in colony forming units S. aureus, except for the Playtex Control, where it was higher. For TSST-1 (μg), the Playtex Control had the highest value. The Fibre 1 and Fibre 11 had a lower value, and the Fibre 8 were non-detectable. The inventors' alginate fibres have a much lower effect on TSST-1 than already existing tampon fibres (compared to Playtex control). This means that they are safe to use in tampon production and will not have a negative effect in regards to TSST-1 for tampon users.

### Example 4: Microbiome Results

Next, the inventors assessed how alginate fibres Fibre 1, Fibre 11, Fibre 8 and Fibre 6 affect the microbiome of the vaginal area.

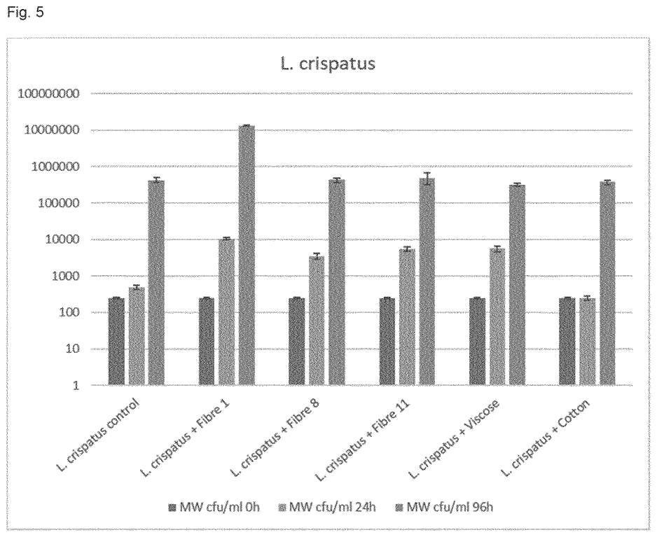

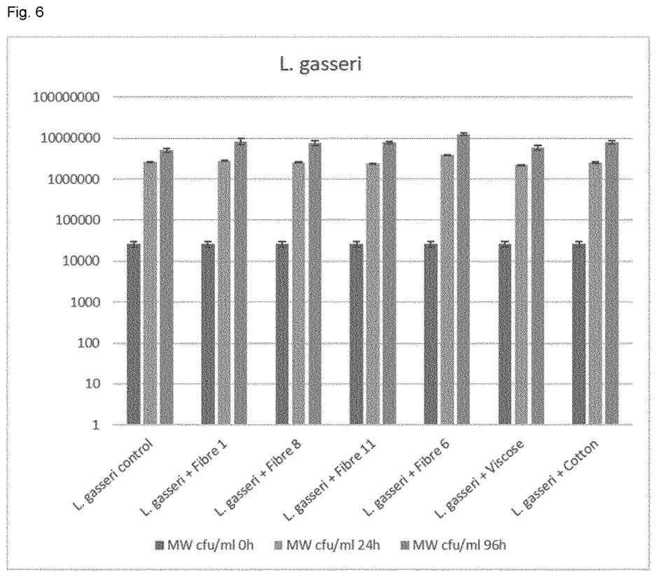

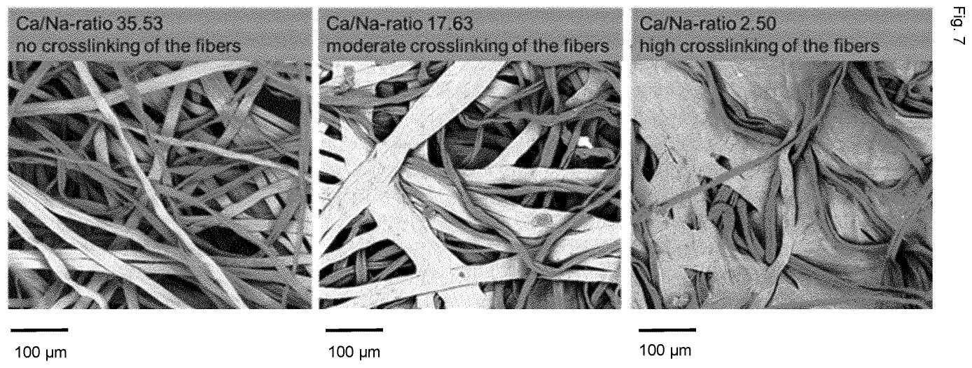

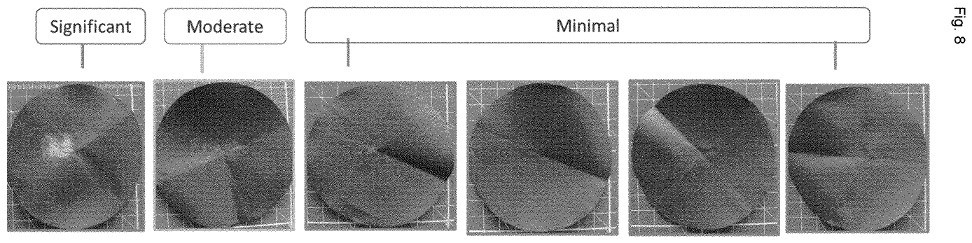

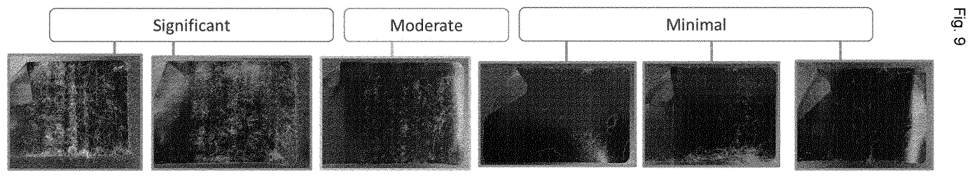

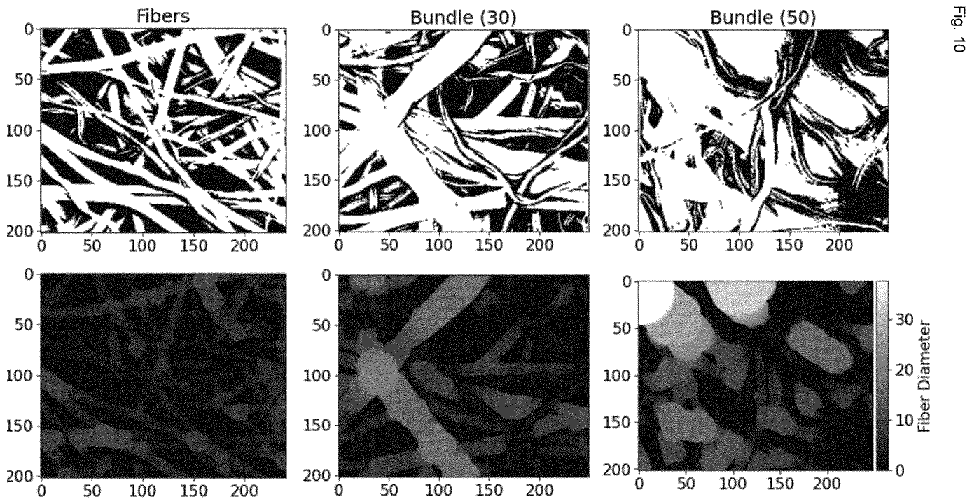

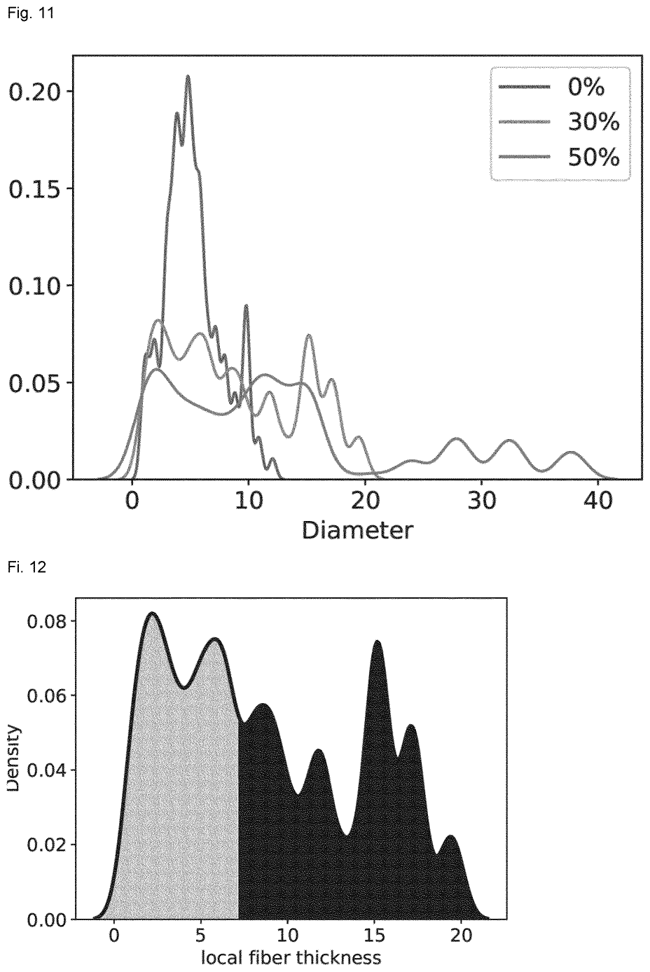

The fibres are tested with two different germs of the vaginal area (Lactobacillus crispatus and Lactobacillus gasseri) incubated for 24 h in liquid culture. After t=0 h, t=12 h and t=24 h, the optical density of the cultures is measured and the cultures are plated with a diluted solution to determine the CFU/ml (colony forming units), see FIGS. 5-6.

The inventors' alginate fibres have no negative effect on the germs. They seem to rather stimulate the growth of L. crispatus in the first 24 h compared to the control.

Viscose seems to inhibit the growth of L. crispatus in the first 24 h, which is however compensated after further incubation. L. gasseri grows very similar to the control regardless of fibre.

These results show that the inventors' tampons would have no negative effects on the microbiome of the vaginal area. On the contrary, they can stimulate the growth of L. crispatus which has a beneficial effect on said microbiome.

### Example 5: Fibre Shedding Prevention

To evaluate the amount of loose fibre that can be detached from the tampon surface (fibre shedding), two methods were developed:
Fibre shedding in saline investigating fibre detachment while tampon is immersed in saline solution.
Dry fibre shedding—assessment of the amount of fibre that can be detached from the surface of a dry tampon.

Commercial tampons were tested to verify if the variance in shedding between different tampons can be measured. Two repeats were conducted for each tampon. A summary of the results is shown below in Table 3.

TABLE 3
Dry shedding and shedding in saline summary
for commercial and alginate tampons.

| Tampon | | | Shedding | Dry |
|---|---|---|---|---|
| Sample | Fibre type | Overwrap | in saline | shedding |
| Tampon 1 | viscose | Yes - plastic | Moderate | Minimal |
| Tampon 2 | cotton | Yes - cotton | Minimal | Moderate |
| Tampon 3 | round viscose/ | Yes - plastic | Significant | Minimal |
| | trilobal viscose | | | |
| Tampon 4 | cotton | Yes - plastic | Minimal | Minimal |
| Tampon 5 | cotton | No | Minimal | Significant |
| Tampon 6 | cotton | No | Minimal | Significant |
| Tampon 7 | cotton/alginate | No | Moderate | Significant |
| Alginate | | | | |
| Tampon 8 | cotton/alginate | No - Alginate | Minimal | Significant |
| Alginate | | surface | | |
| Tampon 9 | cotton/alginate | No | Moderate | Significant |
| Alginate | | | | |

### Example 6: PBS/Overwrap Lamination

Polybutylene succinate (PBS) is thermoplastic and biodegradable polymer that can be produced from renewable sources (fermentation of glucose and sucrose). A number of proof-of-concept prototypes were made, using 50% Fibre 1: 50% cotton blend, cotton spunlace nonwoven (50 g/m²) and a thermoplastic PBS film. To protect tampon core fibres from shedding, 5 cm cotton spunlace overwrap was attached to the ribbon using a layer of thermoplastic perforated PBS film. End of the ribbon, before rolling, was pressed for 30 seconds at 60° C. to attach overwrap similarly as in industrial process. Syngina absorbency of PBS+cotton spunlace overwrap prototypes are comparable to non-overwrap tampon prototype, 431% and 432%, respectively. PBS film, used as lamination layer between ribbon and overwrap, was permeable to Syngina test liquid.

TABLE 4
Syngina results

| | Average tampon weight | Syngina absorbency | | |
|---|---|---|---|---|
| Sample | after conditioning [g] | [g] | [%] | SD [%] |
| Alginate tampon 9 | 2.55 | 11.02 | 432 | 26 |
| Alginate tampon 8 | 2.57 | 9.27 | 361 | 13 |
| Overwrap prototype | 2.65 | 11.41 | 431 | 23 |

In conclusion, lamination of an overwrap with thermoplastic PBS film shows promise as a fibre loss prevention strategy. Minimal shedding was observed in saline and moderate in dry shedding test.

### Example 7: Alginate Gelling—Analysis of Results

Sprayed and non-sprayed prototypes shed significant amount of fibre during the dry shedding test and moderate amount of fibre in saline shedding test. Shedding results show that spraying of 0.31 ml of DI water onto the surface of tampon do not visibly reduce fibre shedding, to compare to unsprayed tampon.

The amount of sprayed DI water (0.31 ml) in not sufficient to prevent shedding. Further increase in the amount of sprayed water significantly reduces the Syngina absorbency below minimum required (<9 g).

Non-sprayed tampons of the invention lost more fibre than the sprayed tampon when tested in saline. It is suspected that spraying with deionised water (DI) water prevents dry shedding by creating a smoother outer layer of cotton fibres joined by gelled alginate fibres.

### Example 8: Alginate Gelling Hydration Method

By applying approx. 0.32 ml of water, prepared prototypes passed the Syngina absorbency test (average Syngina absorbency over 9 g). Increasing the amount of water sprayed on the ribbon before pressing results in a decrease in Syngina absorbency of the tampon.

Prior to tampon formation, deionised water was applied on a 7.2 cm of the ribbon from one end so only the outer surface of the tampon roll is moistened (7.2 cm is circumference of a circle calculated for 23 mm diameter ribbon roll). Temperature during pressing was reduced from 100° C. to 80° C. to stop the water evaporating from the surface during pressing. Spraying triggers gelling of the alginate fibres. Gelling alginate can potentially improve integrity of the tampon surface preventing fibre shedding.

Table 5: Prior to Syngina testing all tampons were unwrapped and conditioned for minimum 3 h at 23° C. and 65% RH

Two sets of sprayed tampon prototypes (3 specimens each) were prepared. 1.08 and 0.32 ml of deionised water was applied on prototype set 1 and 2 respectively to evaluate the effect of spraying volume on Syngina performance. No gel blocking was observed for both high and low spraying volumes although the spray volume does impact absorbency. The required amount of water to achieve target tampon absorbency is around 0.32 ml (see Table 5).

TABLE 5

| Sample - Average DI | Average tampon | | | |
|---|---|---|---|---|
| content added during | weight after | Average absorbency | | |
| spraying [ml] | conditioning [g] | [g] | [%] | SD [%] |
| Tampon 1 - 1.08 ml | 2.68 | 6.95 | 260 | 10 |
| Tampon 2 - 0.32 ml | 2.57 | 9.27 | 361 | 13 |

### CITED PRIOR ART DOCUMENTS

All scientific publications and patent documents cited in the present specification are incorporated by reference herein.

1. A tampon, wherein
a tampon absorption body comprises 25%-100% (w/w) of alginate fibre;
the tampon does not comprise polyolefin fibre;
the alginate is composed of β-(1-4) linked d-mannuronic acid (=“M”) units and β-(1-4)-linked 1-guluronic acid units (=“G”) units, and the alginate is characterized by an M/G ratio (unit/unit) of 0.4-1.5; and
the alginate is present as a mixed salt comprising sodium and calcium ions, and the alginate is characterized by a weight ratio of calcium to sodium between 1-200.

2. The tampon according to claim 1, wherein the tampon absorption body comprises ≥50% (w/w) alginate, particularly the tampon absorption body comprises ≥65% (w/w), ≥70% (w/w), ≥75% (w/w), ≥80% (w/w), ≥85% (w/w), ≥90% (w/w), ≥95% (w/w) alginate, more particularly the tampon absorption body consists of 100% (w/w) alginate.

3. The tampon according to claim 1, wherein the alginate is characterized by an M/G ratio (unit/unit) of 0.45-0.9, particularly the alginate is characterized by an M/G ratio (unit/unit) of ~0.5.

4. The tampon according to claim 1, wherein the alginate is characterized by a weight ratio of calcium to sodium between 2.5-120, particularly the alginate is characterized by a weight ratio of calcium to sodium between 10-50, even more particularly between 17-35, more particularly the alginate is characterized by a weight ratio of calcium to sodium ~25.

5. The tampon according to claim 1, wherein the alginate is characterized by an M/G ratio (unit/unit) of ~0.5 and a calcium to sodium weight ratio of 17-35.

6. The tampon according to claim 1, wherein the tampon comprises a bio-degradable liner.

7. The tampon according to claim 6, wherein the bio-degradable liner is composed of polybutylene succinate.

8. The tampon according to claim 1, wherein the alginate is obtained from algae of the class Phaeophyceae, particularly from algae of a genus selected from the group comprising Macrocystis, Laminaria, Lessonia, Ascophyllum, Saccharina, Sargassum, Fucus.

9. The tampon according to claim 1, wherein the outer surface of the tampon pledget is characterized by a ratio of non-bundled fibre strands to bundled fibre strands of at most 2.3, particularly of at most 1.0.

10. Alginate for use in prevention of toxic shock syndrome (TSS), wherein the alginate is administered in the form of the tampon of claim 1, wherein
the alginate is composed of β-(1-4) linked d-mannuronic acid (=“M”) units and β-(1-4)-linked 1-guluronic acid units (=“G”) units, and the alginate is characterized by an M/G ratio (unit/unit) of 0.4-1.5; and
the alginate is present as a mixed salt comprising sodium and calcium ions, and the alginate is characterized by a weight ratio of calcium to sodium between 1-200.

11. Alginate for use in prevention of vaginal infection, wherein the alginate is administered in the form of the of claim 1, wherein
the alginate is composed of β-(1-4) linked d-mannuronic acid (=“M”) units and β-(1-4)-linked 1-guluronic acid units (=“G”) units, and the alginate is characterized by an M/G ratio (unit/unit) of 0.4-1.5; and
the alginate is present as a mixed salt comprising sodium and calcium ions, and the alginate is characterized by a weight ratio of calcium to sodium between 1-200
particularly wherein the vaginal infection is vaginal yeast infection or infection with E. coli.

12. A method for production of a tampon, said method comprising the steps:
a. providing a longitudinally extending strip of fibre material as a tampon ribbon, wherein the fibre material is a fibre blend comprising 25%-100% (w/w) alginate, and 0-75% (w/w) other bio-degradable fibre;
b. a spraying step;
c. in a rolling step, rolling the tampon ribbon yielding a non-shaped tampon pledget;
d. in a pressing step, pressing the non-shaped tampon pledget via a press to form a shaped tampon pledget;
e. in a head-forming step, forming a head for the shaped tampon pledget using a heated round head and a pointed head shaper to yield a tampon;
characterized in that in the spraying step, one part of the tampon ribbon is sprayed with water, and the water-sprayed part forms the outer surface of the tampon pledget.

13. A method for production of a tampon, said method comprising the steps:
a. providing a longitudinally extending strip of fibre material as a tampon ribbon;
b. a spraying step;
c. in a rolling step, rolling the tampon ribbon yielding a non-shaped tampon pledget, wherein an outer surface of the tampon pledget comprises 10%-100% (w/w) alginate;
d. in a pressing step, pressing the non-shaped tampon pledget via a press to form a shaped tampon pledget;
e. in a head-forming step, forming a head for the shaped tampon pledget using a heated round head and a pointed head shaper to yield a tampon;
characterized in that in the spraying step, one part of the tampon ribbon is sprayed with water, and the water-sprayed part forms the outer surface of the tampon pledget.

14. (canceled)

15. The method according to claim 12, wherein the percentage of water being used per tampon is 5-20% (v/w), particularly wherein the percentage is ~10% (v/w).

16. The method according to claim 12, wherein the temperature of the press in the pressing step is ≤200° C., particularly the temperature of the press in the pressing step is ≤120° C.

17. (canceled)

18. (canceled)

19. The method according to claim 12, wherein the water-sprayed part of the tampon ribbon is ~⅓ of the length of the tampon ribbon.

20. The method according to claim 12, wherein a sodium ion concentration of said water is 0.43-2.2 mol/L.

21. A tampon obtained by the method according to claim 12.

22. The tampon according to claim 21, wherein the tampon is characterized by having no overwrap and wherein the tampon is capable of losing only a medium to low amount of fibre in a saline shedding test.

23. The tampon according to claim 21, wherein the tampon is characterized by having no overwrap and wherein the tampon is capable of losing only a medium to low amount of fibre in a dry shedding test.
